(19) United States
(12) Patent Application Publication
(10) Pub. No.: US 2025/0019405 A1
(43) Pub. Date: Jan. 16, 2025

(54) A Composition and A Method of Rejuvenating DNA and Preventing DNA Damage

(71) Applicant: CHULALONGKORN UNIVERSITY, Pathumwan, Bangkok (TH)

(72) Inventor: Apiwat MUTIRANGURA, Pathumwan (TH)

(21) Appl. No.: 17/995,971

(22) PCT Filed: Apr. 22, 2020
(86) PCT No.: PCT/TH2020/000026

§ 371 Date: Oct. 11, 2022

(51) Int. Cl. C07K 14/47; C12N 15/85

(52) U.S. Cl. CPC C07K 14/47; C12N 15/85

(57) ABSTRACT

The present disclosure relates to a method for rejuvenating DNA and/or preventing DNA damage within a nucleus of a cell. The method generally comprises administering a reagent having a vector containing a polynucleotide sequence as setting forth in SEQ ID No. I or SEQ ID No. 2 encoding for a peptide into the cell, the vector being configured to express the encoded peptide within the cell; and overexpressing the encoding encoded peptides, wherein the overexpressed peptides form one or more complexes, within the cell, capable of rejuvenating DNA and preventing damage of DNA in the nucleus of the cell.

In response to the “Notification to Comply with Requirements for Patent Application” issued on Mar. 14, 2023, a sequence listing in a “txt” format is hereby submitted.
sequence listing file name: “petition_revival_SQ_17995971”
sequence listing file size: 2493 bytes

Applicant states there is no new matter being introduced to this submitted sequence listing nor to the specifications. This submitted sequence listing is same as the sequence listing (txt format) submitted on Oct. 11, 2022.

### TECHNICAL FIELD

The present disclosure relates to a composition and a method for rejuvenating DNA and preventing DNA damages in cells. More particularly, the disclosed composition and method facilitates formation of one or more biological complexes inside the cells, preferably within the nucleus, to generate significant stability of DNA leading to resistance against DNA damaging agents or limiting DNA damage in the nucleus. The mentioned biological complexes are generally depleted in the elderly due to aging. The present disclosure also covers any artificial mean to facilitate the yield of Box A of HMGB1 peptide in nucleus or transport of Box A of HMGB1 into nucleus to produce physiologic replication-independent endogenous DNA double-strand breaks (Phy-RIND-EDSBs) or youth-associated genomic-stabilizing DNA gaps (Youth-DNA-GAPs) thus conferring the host genome with enhanced resistance against DNA damage.

### BACKGROUND

Endogenous DNA damage relating to depletion of epigenetic marks has been hypothesized to cause health deterioration in the elderly and many noncommunicable disease (NCD) patients (1). Moreover, DNA damage can also be caused by a different externally and internally presented agents such as heat, ultraviolet light, free radicals, methylating agents and other mutagenic compounds. DNA damage can lead to mutations causing carcinogenesis or congenital defects. To prevent mutation, cells possess DNA damage response (DDR) to detect, signal, halt cell proliferation and/or repair cell's DNA damage. However, excessive interference from DDR may drive cells into metabolic dysfunction, poor growth, cellular aging including senescence and death or apoptosis (2,3).

Numerous researches have been conducted worldwide aiming to treat various diseases via manipulation of DNA repairment in cells. For instance, U.S. Pat. No. 9,359,605 teaches a method of treating solid tumor lung cancer by inhibiting BRCA2 and RAD51, which are DNA double-strand break repair proteins. Similarly, International patent application no. PCT/EP2014/057904 describes a polynucleotide based-molecule capable of inhibiting poly-(ADP-ribose) polymerase (PARP) mechanism in the treatment of cancer. Adam et al. further suggest possible ways to prevent mitochondrial dysfunction in a human subject through administering extract of a fruit of genus Elaeis in International patent application no. PCT/US2014/015110.

Considering that genomic instability in cells may be driven by decrease of epigenomic modifications (1) as well, it is likely that any attempt to promote epigenetic editing like adding epigenetic marks in the cells may reduce DNA damage. Through proper epigenetic modification, the deterioration of cellular function may be restored (1). One of the epigenetic marks known to be effective against DNA damage is Phy-RIND-EDSBs or Youth-DNA-GAPs (1,4). Therefore, there exists a need to find ways to facilitate formation of Youth-DNA-GAPs, which shall inevitably improve clinical conditions of diseases, associated with biological aging DNA and/or accumulated DNA damage.

### SUMMARY

The present disclosure aims to provide a composition capable of rejuvenating cells and/or preventing damage of cellular DNA. More particularly, the disclosed composition facilitates expression or transfection of Box A of HMGB1 protein in the cells administered with the disclosed composition to attain improved genomic stabilization effect.

Further object of the present disclosure is directed to a composition for rejuvenating biological aging cells, restoring cell growth and healing process of subjects suffering from potential DNA damage such as burn injury or having low levels of Youth-DNA-GAPs including elderly and individuals with diabetes mellitus (DM).

Still another object of the present disclosure is to offer a method for preventing DNA damage of a nucleus of a cell. In more particular, the method includes expression and/or presentation of molecular engineered HMGB1 protein particularly Box A domain such that one or more Youth-DNA-GAPs can be formed within the genome to boast resistance against DNA damage formation.

One aspect of the present disclosure refers to a vector capable of expressing a peptide in a cell for preventing damage of DNA in a nucleus of the cell comprising a polynucleotide sequence as setting forth in SEQ ID No. 1 or SEQ ID No. 2 encoding for the peptide.

One major aspect of the present disclosure refers to a method for producing Youth-DNA-GAPs to preventing DNA damage within a nucleus of a cell comprising transfecting the cell with a reagent comprising a peptide as setting forth in SEQ ID No. 3 and/or a vector containing a polynucleotide sequence as setting forth in SEQ ID No. 1 or SEQ ID No. 2 encoding for the peptide into the cell. Preferably, the vector is being configured to express the encoded peptide within the cell subsequent to the transfecting step that the encoded peptides form one or more complexes including namely Youth-DNA-GAPs within the cell capable of preventing damage of DNA in the nucleus of the cell.

Accordingly, the disclosed method may further comprise overexpressing the peptide encoded by the polynucleotide sequence resided in the transfected vector.

According to further embodiments of the disclosed method, the prevention of DNA damage is obtained by way of reducing DNA damage response.

In several embodiments, the prevention of DNA damage is obtained by way of increasing cellular resistance against DNA damaging agent.

Another aspect of the present disclosure relates to a method for improving healing of wounded tissue of a subject comprising the steps of bringing a reagent comprising a peptide as setting forth in SEQ ID No. 3 and/or a vector containing a polynucleotide sequence as setting forth in SEQ ID No. 1 encoding for the peptide into contact with the wounded tissue consisting of multiple cells; and transfecting the cells of the wounded tissues with the peptide and/or the vector, the vector being configured to express the encoded peptide within the cell subsequent to the transfecting step, wherein the encoded peptides form one or more complexes, within the cell, capable of improving healing of the wounded tissue by way of enhancing growth of the cells.

For a number of embodiments, the subject is a DM patient, who generally tends to have lower Youth-DNA-GAPs formed within the cells thus suffering from diminished healing rate for wounded tissues. The disclosed method enhances healing rate of the wounded tissues in these subjects by way of forming the Youth-DNA-GAPs.

For more embodiments, the wounded tissue is resulted by burn injury.

Further aspect of the present disclosure is directed to a topically or systemic applicable pharmaceutical composition for rejuvenating DNA to reduce DNA damage. The composition generally comprises a vector comprising a polynucleotide sequence as setting forth in SEQ ID No. 1 or SEQ ID No. 2 encoding for a peptide.

According to another aspect of the present disclosure, a method of rejuvenating aging cells, preventing DNA damage and enhancing healing process of a mammal is disclosed. Preferably, the method comprises administering topically or systemically a reagent having an expression vector containing a polynucleotide sequence as setting forth in SEQ ID No. 1 or SEQ ID No. 2 encoding for a peptide. To enhance insertion or intake of the expression vector by the targeted cells, the expression vector may be linked to a carrier such as cell-penetrating peptides, nanoemulsion, etc.

More aspect of the present disclosure includes A pharmaceutical composition for rejuvenating DNA and/or reducing DNA damage in a mammal cell comprising a biological component being one of (i) a vector comprising an expressible polynucleotide sequence as setting forth in SEQ ID No. 1 or SEQ ID No. 2 encoding for a peptide or (ii) a peptide as setting forth in SEQ ID No. 3; and a carrier system being chemically linked to the biological component to facilitate entering of the biological component into the cell upon bringing the composition into contact with the cell, wherein carrier system is a cell-penetrating peptide, nanoemulsion, etc.

### BRIEF DESCRIPTION OF DRAWINGS

FIG. 1 are graphs showing low levels of Youth-DNA-GAPs, EDSBs in elderly individuals and in biologically aging cells example in cells of DM patients that (A) presents correlation between EDSBs and age, (B) presents the level of each EDSB in individuals without DM and with DM, (C) presents the levels of EDSBs in age-matched and sex-matched pairs between individuals without DM (normal) and with DM, the average levels of EDSBs are shown as histograms with error bars representing SEM.

FIG. 2 illustrate Box A of HMGB1 producing Youth-DNA-GAPs which are results of HMGB1 restriction enzyme activity in producing DSBs. (A) is a graph showing increased EDSB percentage of the HeLa DNA reacted to HMGB1 at different ratio with or without T4 polymerase. (B) is a graph showing HMDNA of HK2 and HeLa cells reacted to HMGB1-generated EDSB, more EDSBs being generated in DNA incubated with HMGB1. The experiment was performed in triplicate, and scale bars represented standard error.

FIG. 3 are picture showing Box A of HMGB1 producing Youth-DNA-GAPs. The electro-micrograph of HK-2 cells show the results of DNA damage in-situ ligation followed by proximity ligation assay (DI-PLA) with cells transfect with (A) HMGB1 and (B) Box A emitting positive signals while (C) transfected with Box B, (D) transfected with Box BC, (E) transfected with scrambled sequence control plasmids and (F) without any transfection showing no signal.

FIG. 4 are graphs being indicative of generation of Youth-DNA-GAPs by HMGB1 through Box A of HMGB1 and showing results about the percentage of EDSB input in DNA immunoprecipitation (DIP) that (A) relates to HEK293 and HeLa cell lines in the presence of 8-hydroxy-2′-deoxyguanosine (8-OHdG), (B) relates to HEK293 cells transfected with Box A, HMGB1 and control plasmid in the presence of 8-OHdG and (C) relates to HEK293 cells transfected with Box A, HMGB1 and control plasmid in the presence of 7-methylguanosine where the values are shown as box plots, with the boxes representing interquartile ranges (25th to 75th percentile) and median lines representing the 50th percentile (the whiskers represent minimum and maximum values. *P<0.05, **P<0.01, ***P<0.0001)

FIG. 5 are graphs being indicative of potential application of Box A of HMGB1 in reducing DNA damage and showing results about 8-OHdG levels (8-OHdGs ng/ml) and apurinic/apyrimidinic site (AP-site) levels (AP-sites/100,000 bp) in HMGB1- and Box A-overexpressing HEK293 and HK-2 cells (n=9 each) with (A) is 8-OHdGs and (B) is AP-sites in HMGB1- and Box A-overexpressing cells, respectively (*P<0.05, **P<0.01, ***P<0.0001).

FIG. 6 shows the results of Box A of HMGB1 in reducing that (A) is compiled Western blotting gel pictures with respect to expressed levels of p-ATM (Ser1981), p53, p21, p16^{IN4A}, and γH2AX after HK2 cells treated with HMGB1, Box A or PC at 2,500 ng/μl for 48 hours (blots were reprobed with β-actin to confirm equal sample loading) and (B) is a graph showing corresponding results of (A) with the types of protein plotting against relative protein level (data were compared by one-way ANOVA and Dunnett's multiple comparisons test with *P value <0.05, ** P value <0.01, ***P value <0.001).

FIG. 7 are graphs showing results about Box A of HMGB1 in promoting cellular resistance to DNA-damaging agents in the presence of Box A and/or HMGB1 where (A) relates to outcome obtained for cells transfected with scrambled plasmid, Box A and HMGB1 treated with H₂O₂ for 24 hours, (B) relates to outcome obtained for cells transfected with scrambled plasmid, Box A and HMGB1 treated with MMS for 24 hours where values are the mean±SD of 12 independent assays. Values were compared by one-way ANOVA and Dunnett's multiple comparisons test (*P value <0.05, ** P value <0.01, *** P value <0.001).

FIG. 8 showing results about the effect of Box A of HMGB1 in promoting cell growth where graphs (A), (B), (C) and (D) respectively presenting data about cell proliferation in 4 days MTT assay upon overexpression of Box A in HEK 293 cell, HMGB1 in HEK 293 cell, Box A in HK-2 cell and HMGB1 in HK-2 (C and D) with (n=9 each), *P<0.05, **P<0.01, ***P<0.0001

FIG. 9 is (A) images and (B) a graph showing results about the effect of Box A of HMGB1 in promoting healing of on wound closure in diabetic rats divided into groups, where wound areas of each group were measured using the NIH ImageJ analyzing tool on days 3, 5, 7, 10, and 14 and compared to on the measurements obtained at day 0, with the wound closure rate being significantly enhanced in the Box A of H HMGB1 plasmid-treated group compared to the plasmid control-treated or NSS-treated control group (*P<0.05, **P<0.01, and ***P<0.001, N=8 each group).

FIG. 10 showing results about the effect of Box A of HMGB1 in promoting healing and contracture of wound from second degree, burn injury, where (A) are Images of second-degree burn wounds in rats treated daily with Box A of HMGB1 and control rats treated with normal saline solution with the wounds treated with Box A of HMGB1 exhibited greater contraction and less inflammation than the control group, and (B) is a graph further revealing significantly enhanced healing effect of Box A of HMGB1 towards second degree burnt wound in treated group compared with the control group. P<0.001, *P<0.05, **P<0.01, and ***P<0.001, N=8 each group.

FIG. 11 showing results about the effect of Box A of HMGB1 in reversing aging process with HK2 cells being pretreated with etoposide 2.5 μM for 72 h to induce cellular senescence followed by transfection with BoxA and scramble control plasmids and incubated for 48 h, where (A) are bright field images captured to show cell morphology and density (Scale bar=50 μm) and (B) is Western blotting gel pictures illustrating expression levels of p16^{INK4A} and γH2AX in the different pretreated HK2 cells while β-actin was used for reprobing to confirm equal sample loading.

FIG. 12 is a graph showing results of HK2 cells exposed to DNA damaging agent, H₂O₂, treated with a cell-penetrating peptide IMT-P8-BoxA peptide and control indicating that IMT-P8-BoxA in promoting cellular resistance to DNA-damaging agents and preventing H₂O₂-induced DNA damage, where HK2 were pretreated with IMT-P8-BoxA peptide prior incubated cells with H₂O₂ for 24 h then the survival rate was evaluated by MTT assay after 48 h and compared by one-way ANOVA, Dunnett's multiple comparisons test (*P<0.05, **P<0.01, ***P<0.001).

FIG. 13 shows the SEQ ID No.1 of DNA sequence of BoxA of HMGB1, the SEQ ID No.2 of DNA sequence of HMGB1 and SEQ ID No. 3 of the peptide sequence of Box A of HMGB1.

FIG. 14 is a flowchart showing one embodiment of the disclosed method for rejuvenating DNA and preventing DNA damage.

### DETAILED DESCRIPTION

The present disclosure may be embodied in other specific forms without departing from its structures, methods, or other essential characteristics as broadly described herein and claimed hereinafter. The described embodiments are to be considered in all respects only as illustrative, and not restrictive. The scope of the disclosure is, therefore, indicated by the appended claims, rather than by the foregoing description. All changes that come within the meaning and range of equivalency of the claims are to be embraced within their scope.

The term “polynucleotide” or “nucleic acid” as used herein designates mRNA, RNA, cRNA, cDNA or DNA. The term typically refers to oligonucleotides greater than 30 nucleotide residues in length.

The term “gene” as used herein may refer to a DNA sequence with functional significance. It can be a native nucleic acid sequence, or a recombinant nucleic acid sequences derived from natural source or synthetic construct. The term “gene” may also be used to refer to, for example and without limitation, a cDNA and/or an mRNA encoded by or derived from, directly or indirectly, genomic DNA sequence.

The term “complex”, “biological complex” and “Youth-DNA-GAPs” are used interchangeably throughout the specification, unless mentioned otherwise, referring to an epigenetic biomarker in cell for genomic stability.

The term “an aging cell” and “aging cells” as used herein may refer to cells with deterioration of functional characteristics due to aging process or senescence induction or accumulation of DNA damage. More specifically, “an aging cell” and “aging cells” refer to the cells with Youth-DNA-GAPs lower than 0.3% EDSB PCR of control, and/or accumulation of senescence-associated β-galactosidase cells more than 50%, and/or accumulation of DNA damage in which having more than 3.5 γ-H2AX foci/cell or 8-OhDG more than 7 8-OHdG/10⁶ dG.

In accordance with one of the aspects of the present disclosure, a method for preventing DNA damage within a nucleus of a cell is disclosed. Preferably, the method comprises the steps of administering a reagent having a vector containing a polynucleotide sequence as setting forth in SEQ ID No. 1 or SEQ ID No. 2 encoding for a peptide into the cell that the vector is configured to express the encoded peptide within the cell; and overexpressing the encoded peptides in the cells. Preferably, the overexpressed peptides form one or more complexes (or Youth-DNA-GAPs), within the cell, capable of preventing damage of DNA in the nucleus of the cell. In more specific, the peptides encoded respectively by SEQ ID No. 1 or SEQ ID No. 2 are Box A of HMGB1 protein and HMGB1 protein. One in the relevant field shall appreciate the fact that the polynucleotide sequence setting forth in SEQ ID No. 1 and SEQ ID No. 2 can be further modified for enhancement like better expression or more compatible with a specific host cells type. These modifications may render the modified polynucleotide sequence retaining merely around 70 to 90% of the sequence setting out in SEQ ID No. 1 and SEQ ID No. 2. Preferably, such modifications shall not depart from the scope covered by the present disclosure about a Box A of HMGB1 and/or HMGB1-based composition, method or reagent for preventing DNA damage, accelerated external wound healing, treatment of noncommunicable disease related to low yield of Youth-DNA-GAPs in a subject, etc.

For a number of embodiments, the administering step may refer to applying the reagent to a subject via the topical, enteral and/or parenteral route though topical application is more preferable due to less invasive nature. The reagent of the present disclosure may adapt to different forms according to the administering route to attain the desired outcome. In those embodiments where the reagent to be administered topically, the reagent comprises a vector containing a polynucleotide sequence as setting forth in SEQ ID No. 1 or SEQ ID No. 2 encoding respectively for a peptide of Box A of HMBG1 protein or HMBG1 protein Particularly, the vector, as to an expression plasmid used, is preferably nano-coated in the form of nanoemulsion to facilitate its adsorption into the cells or tissues around the external wound thus expressing the Box A of HMBG1 protein or the HMGB1 protein through the vector within the cell and subsequently forming the complex capable of preventing DNA damage. More specifically, the present disclosure found that HMGB1 or Box A of HMGB1 is able to generate Youth-DNA-GAPs in cells equipping the cells with great resistance against wide range of DNA damaging agents. The known HMGB1 protein's deoxyribose phosphate lyase activity and ability to bend DNA may play a role in the yield of Youth-DNA-GAPs. The HMGB1 gene contains two DNA-binding domains (box A and box B) and an acidic tail. Thus far, the present disclosure only finds that Box A protein or HMGB1 protein possessing the Box A domain appears to have the competence to confer the cells with resistance against DNA damaging agents and limiting the DNA damage. Accordingly, the vector employed in the disclosed method of preventing DNA damage within a nucleus of a cell may comprise one or more regulatory sequences operable with Seq ID No. 1 or Seq ID No. 2. For a number of the embodiments, peptide transfection system such as cell-penetrating peptide can be used as well for transportation of the vector into the cells.

According to further aspect of the present disclosure, a method for preventing DNA damage within a nucleus of a cell is disclosed. The method essentially comprises the steps of transfecting the cell with a reagent comprising a peptide as setting forth in SEQ ID No. 3 and/or a vector containing a polynucleotide sequence as setting forth in SEQ ID No. 1 or SEQ ID No. 2 encoding for the peptide into the cell. Preferably, the vector is being configured to express the encoded peptide within the cell subsequent to the transfecting step. More preferably, the encoded peptides form one or more complexes, including namely Youth-DNA-GAPs, within the cell capable of preventing damage of DNA in the nucleus of the cell. In several embodiments, the vector may possess a promoter region which only initiates transcription and translation of the encoded peptides in the presence of a promoting entity such that the expression of the peptides can be regulated at a predetermined time, period, level and/or cells of specific tissue.

For a number of embodiments, the method of preventing DNA damage may further include overexpressing the encoded peptides through transfecting the cells with a predetermined number of the expression vector or upregulating expression of the peptides using a predetermined concentration of the promoting entity. Also, in some embodiments, the prevention of DNA damage and reducing DNA damage response is obtained by way of producing Youth-DNA-GAPs as shown in some of the examples given hereinafter. Alternatively, increasing cellular resistance against DNA damaging agent can also be facilitated by way of producing Youth-DNA-GAPs.

For another aspect, the present disclosure refers to a method for improving healing of wounded tissue of a subject comprising the steps of bringing a reagent comprising a peptide as setting forth in SEQ ID No. 3 and/or a vector containing a polynucleotide sequence as setting forth in SEQ ID No. 1 or SEQ ID No. 2 encoding for the peptide into contact with the wounded tissue consisting of multiple cells; and transfecting the cells of the wounded tissues with the peptide and/or the vector. Likewise, the vector is being configured to express the encoded peptide within the cell subsequent to the transfecting step. Particularly, the encoded peptides form one or more complexes, within the cell, capable of improving healing of the wounded tissue by way of correcting healing delay in DNA damaged cells or biologically aging cells. This disclosed method can significantly enhance healing rate of the wounded the subject when the subject is a diabetic mammal or DM patient. For some embodiments, the wounded tissue is resulted by burn injury.

Pursuant to further aspect of the present disclosure, a pharmaceutical composition for preventing DNA damage is disclosed. The composition comprises a vector containing a polynucleotide sequence as setting forth in SEQ ID No. 1 or SEQ ID No. 2 encoding for Box A protein or HMGB1. The vector may be incorporated with regulatory sequences or regions for regulating expression of the encoded proteins. The vector may be encapsulated or linked with a carrier system such as cell-penetrating peptide or nanoemulsion for transporting the vector effectively into the cells. The composition can be either administrated topically or systemically depending on the embodiment adapted. The present disclosure found a significant inversed association between Youth-DNA-GAPs and elderly individuals or DM patients, both of whom are known to have biologically aging DNA. In turn, DNA damage is commonly found in age-associated noncommunicable diseases (NCDs). Specifically, the Youth-DNA-GAPs prevent DNA damage. With the reduction of Youth-DNA-GAPs in elderly individuals and diabetic subjects, these subjects suffer increased endogenous DNA damage and elevated DDR leading to unhealthy cellular function. The disclosed composition introduces the plasmid or vector for expressing, or more preferably overexpressing, the Box A of HMGB1 protein and/or the HMGB1 protein, which was found to be a means for generating Youth-DNA-GAPs in cells.

Furthermore, the present disclosure demonstrates that Box A of HMGB1 stabilized the human genome better than HMGB1. Inventors of the present disclosure believe that Box A of HMGB1 peptide can enter the nucleus and form Youth-DNA-GAPs thereof. Box A of HMGB1 peptide possesses all known roles of HMGB1 that are needed in Youth-DNA-GAP formation. In addition to entering the nucleus and producing Youth-DNA-GAPs, at Box A, HMGB1 is known to have other roles interacting with different extracellular and intracellular enzymes and components. The present disclosure assumes that divided role of HMGB1 protein has resulted its lowered efficiencies in the production of the complexes or Youth-DNA-GAPs. In connection to that, some embodiments of the various aspects of the present disclosure have deliberately excluded expression of the Box B and C terminals of the HMGB1 proteins but rather specifically utilized the Box A of HMGB1 in preventing the DNA damage and/or rejuvenating the damaged cells in addition to the extracellular role of Box A of HMGB1.

As in the foregoing description, the inventors further revealed that the formed complex can be utilized for monitoring and preventing genomic instability, which are causes of health deterioration in a number of diseased states. Hence, further aspect of the present disclosure is drawn to a method of rejuvenating cells and enhancing wound healing rate on a biological aging subject comprising administering topically or systemically a reagent having a vector containing a polynucleotide sequence as setting forth in SEQ ID No. 1 or SEQ ID No. 2 encoding for a peptide. Preferably, the reagent initiates overexpression of the encoded peptides in the biologically aging cells that the overexpressed peptides subsequently lead to formation of the Youth-DNA-GAPs or complexes, which improve genome stability in the affected cells and prohibit at least one or more of the DDR enzymatic reaction. Particularly, Phy-RIND-EDSB or Youth-DNA-GAP is a unique type of epigenomic marker in humans that this epigenomic marker becomes progressively decreased in elderly and DM patients. The present disclosure revealed that HMGB1 or Box A of HMGB1 protein is an effective tool in producing Youth-DNA-GAPs within cells. Ensuing formation of the Youth-DNA-GAPs, the topically applied composition is able to increase the stability of the DNA strand in long-distance cis, reduce endogenous DNA damage as well as the DDR, and improve cellular resistance to DNA-damaging agents. As such, the disclosed composition rejuvenates cells and promotes wound healing in the biologically aging subject through overexpressing Box A of HMGB1 and/or HMGB1. Preferably, the subject is a biologically aging mammal. The disclosed composition can be used to address health problems in age related NCDs including the elderly and diabetic subjects about poor cell growth, cellular senescence and healing delays.

The inventors of the present disclosure discovered the genomic stabilizing biomarker, Youth-DNA-GAPs or the complex, potentially formed in the presence of HMGB1 protein or Box A of the HMGB1 protein. It is crucial to note that Youth-DNA-GAPs are epigenetic marks and not DNA damage. Epigenetic marks are produced by cellular enzymatic activity. Epigenetic marks such as Youth-DNA-GAPs are shown hereby by the examples provided hereinafter benefiting to wounded and/or aging cells. The disclosed examples demonstrated also that Youth-DNA-GAPs produced by HMGB1 or Box A of HMGB1 stabilize the genome; therefore, Youth-DNA-GAPs are epigenetic marks.

Still, another aspect of the present disclosure is about a pharmaceutical composition for generating Youth-DNA-GAPs preventing DNA damage, increasing resistance against DNA damage, enhancing cell growth, improving healing of wounded tissue, reducing endogenous DNA damage, and/or reducing DNA damage response in a mammal is disclosed. The composition comprises a peptide comprising at least 70% of amino acid sequence as setting forth in SEQ ID no. 3; and a carrier chemically linked to the peptide and being configured to bring the linked peptide into cells of a predetermined tissue type of the mammal to attain the beneficial outcome towards the mammal as mentioned in the setting forth. The carrier can be cell-penetrating peptide, nanoemulsion and the like which allows the linked peptide to exert the mentioned one or more the mentioned beneficial outcome in the cells brought into contact with the disclosed composition in an immediate or almost immediate fashion compared to other disclosed embodiments employing expression vector.

The following example is intended to further illustrate the disclosure, without any intent for the disclosure to be limited to the specific embodiments described therein.

### Example 1

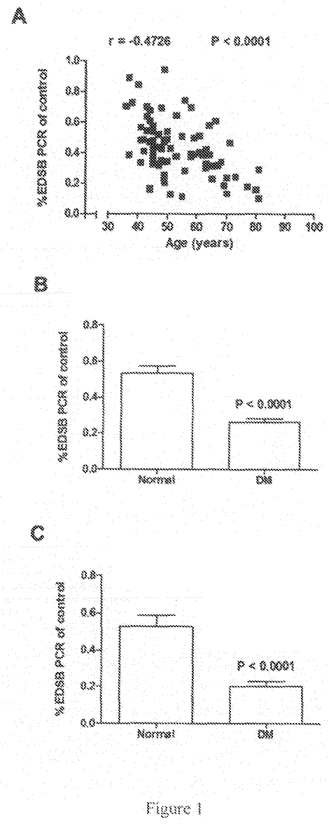

Low levels of Youth-DNA-GAPs are reported in biology aging individuals, the elderly and individuals with DM. Particularly, Hemoglobin A1C (HbA1C) levels were assessed in 120 patients, who were then classified into non-DM (80 samples) and DM (40 samples) groups. All subjects were recruited from the Tambon Health Promoting Hospital service, Nakhon Si Thammarat, Thailand, between 2015 and 2016. The participant ages were between 15 and 80 years. All subjects voluntarily participated in the study. The study was reviewed and approved by the Ethics Clearance Committee on Human Rights Related to Research Involving Human Subjects at Walailak University, Nakhon Si Thammarat, Thailand. Written informed consent was obtained from each participant. In yeast, Youth-DNA-GAPs are chronologically reduced in aging yeast and lowering Youth-DNA-GAPs drives the biological aging process (4). The present disclosure found low Youth-DNA-GAP levels in elderly participants (r=−0.4726, P<0.0001) (FIG. 1A). Diabetic patients were known to have an accelerated cellular aging process (5). The present disclosure also found lower levels of Youth-DNA-GAPs in the WBCs of diabetic patients than in the WBCs of nondiabetic patients (FIG. 1B and sex age adjusted FIG. 1C). Therefore, a decreased level of Youth-DNA-GAPs in humans was found in populations that are known to have biological aging of DNA. These data led the present disclosure to hypothesize that as in yeast, the reduction in Youth-DNA-GAPs promotes DNA damage and a subsequent deterioration of cellular function.

### Example 2

The present disclosure found that HMGB1 generates Youth-DNA-GAPs because HMGB1 protein possesses lyase activity. To produce HMGB1 protein, HMGB1 cDNA (NM_001313893.1) in the pRSET A vector (Thermo Fisher Scientific, MA, USA) was generated (6). Vector construction was performed by GeneArt™ Gene Synthesis (Thermo Fisher Scientific, MA, USA). Sequence fidelity was confirmed by Sanger sequencing. The HMGB1 vector was then transformed into BL21(DE3)pLysS competent cells (Promega, WI, USA) for protein production.

DNA from two human immortalized kidney cell lines, HEK293 and HK-2, and a cervical cancer cell line, HeLa cells were incubated with 2 μg of HMGB1 protein in 1× CutSmart® buffer (New England Biolabs, MA, USA) in a total volume of 50 μl at 37° C. for 16 h. Two micrograms of HeLa DNA incubated with purified EGFP protein or AluI (New England Biolabs) was used as a control. Moreover, the dosage of DNA to HMGB1 protein ratio varied from 5:1, 4:1, 3:1, 2:1 and 1:1.

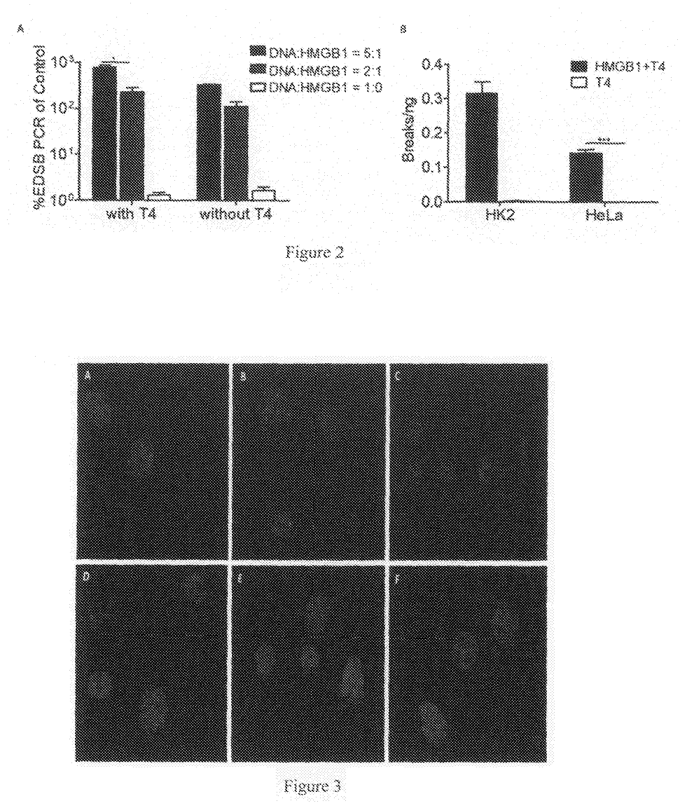

To measure Phy-RIND-EDSBs or Youth-DNA-GAPs caused by HMGB1 lyase activity, EDSB PCR was performed as previously described (7) The present disclosure found that the purified HMGB1 protein can digest DNA in a dosage-dependent manner (FIGS. 2A-D). The majority of the ends of HMGB1-generated DSBs were blunt (FIG. 2C).

### Example 3

The present disclosure found that Box A of HMGB1 generates Youth-DNA-GAPs and consequently colocalizes with Youth-DNA-GAPs. The present disclosure determined colocalization between a Youth-DNA-GAP and a protein from expression plasmids with a DI-PLA (8). Full-length human HMGB1, Box A, Box B, Box BC, and scrambled sequence control (PC) expression plasmids were used in this study. The present disclosure transformed a commercial pcDNA3.1 Flag insert expression vectors (Invitrogen, Carlsbad, U.S.A.) into Escherichia coli (DH5α) host cells. Plasmid DNA isolation was performed using a Qiagen Plasmid Miniprep Kit (Qiagen, Switzerland) according to the manufacturer's instructions. Cells were transfected with plasmids (final concentration of plasmids, 2,500 ng/ml) using Lipofectamine 3000 (transfection reagent) (Invitrogen, Carlsbad, U.S.A.) and incubated in incubator culture for 24-48 hours.

Further, the present disclosure performed DI-PLA between Flag and DSB as previously described (8). The DI-PLA was performed by Duolink® In Situ Orange Starter Kit Mouse/Rabbit (DUO92102) (Sigma-Aldrich®, Missouri, USA) following the manufacturer's protocol. The samples were incubated for 15 min at room temperature before analysis in a fluorescence or confocal microscope using 20× and 40× objective lenses.

Plasmid protein localization at a DSB was observed with the DI-PLA technique at each of the red spots (FIG. 3). The DI-PLA results showed that the HMGB1 and Box A bound DNA near each Youth-DNA-GAP whereas the Box B and BC molecules did not. Positive signals in cells with HMGB1 (FIG. 3A) and Box A plasmids (FIG. 3B) were observed. No DI-PLA signal was revealed in cells transfected with Box B (FIG. 3C), Box BC (FIG. 3D), control plasmids (FIG. 3E) and untransfected cells (FIG. 3F).

### Example 4

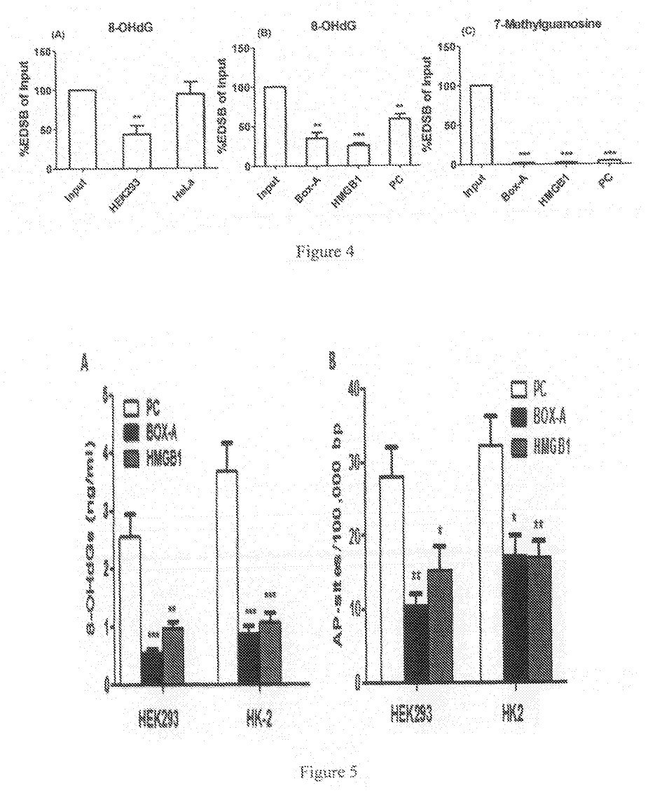

The present disclosure concludes that Box A of HMGB1 generates Youth-DNA-GAPs to prevent DNA damage. If Youth-DNA-GAPs prevent DNA damage, Youth-DNA-GAPs and DNA damage should rarely coexist. The present disclosure performed DIP (9) with an antibody against DNA damage and compared the concentration of EDSBs of DIP DNA with that of input DNA. First, prepared HMWDNA from full-length human HMGB1, Box A, Box B, Box BC, and PC expression plasmids transfected cells. Second, EDSB linker ligated HMWDNA was prepared as previously described (7). Then, EDSBs of DIP DNA were compared with input DNA using EDSBPCR protocol as previously described (7). FIG. 4A demonstrates % EDSBPCR of input of HeLa and HEK293 cell lines. Then, the present disclosure measured the coexistence in cis between 8-OHdG or 7-methylguanosine and EDSBs of cells transfected with Box A of HMGB1 and HMGB1 expression plasmids and a negative control plasmid (FIGS. 4B and C). Genomes containing DNA damage of all cells tested lacked EDSBs significantly in which the genomes containing DNA damage of cells transfected with Box A of HMGB1 and HMGB1 expression plasmids had fewer EDSBs than those transfected with the negative control plasmid (FIGS. 4B and C). Therefore, Box A of HMGB1 and the HMGB1 expression plasmid increased the proportion Youth-DNA-GAPs in the cells.

### Example 5

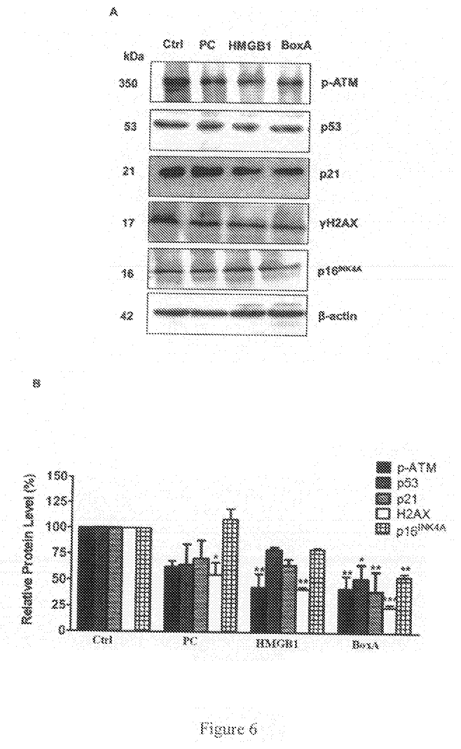

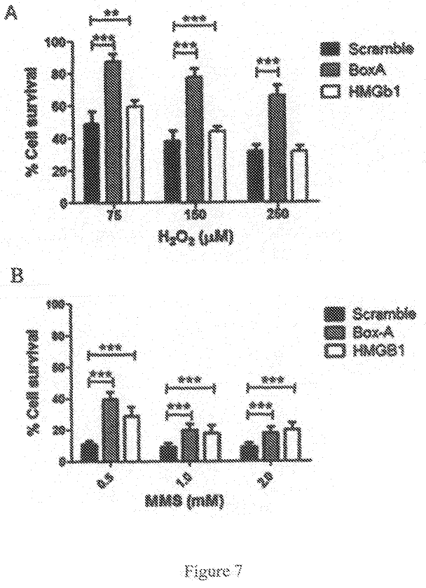

The present disclosure discovered that Box A of HMGB1 can reduce endogenous DNA damage. Specifically, the DNA from full-length human HMGB1, Box A, and PC expression plasmids transfected cells was extracted by the phenol-chloroform method and resuspended in sterilized dH₂O. Subsequently, 8-OHdG levels in DNA were measured using an OxiSelect™ Oxidative DNA Damage ELISA Kit (Cell Bio Labs, Inc., San Diego, U.S.A.). The AP site levels were determined by an OxiSelect™ Oxidative DNA Damage Quantitation Kit (Cell Bio Labs, Inc., San Diego, U.S.A.). The present disclosure transfected HMGB1 and Box A expression plasmids into HEK293 and HK2 cell lines and found that both plasmids caused the reduction in several types of endogenous DNA damage including 8-OHdG and the AP-site (FIG. 5A-4D). Therefore, the endogenous DNA damage reduction role of HMGB1 belongs to the Box A domain.

### Example 6

The present disclosure found that Box A of HMGB1 reduces DDR. Particularly, the protein lysates from full-length human HMGB1, Box A, and PC expression plasmids transfected cells were prepared using RIPA buffer (Sigma Chemical, St. Louis, MO, USA) and protease inhibitor mixture (Pierce Biotechnology, Rockford, IL, USA) and analyzed by a BCA protein assay kit from Pierce Biotechnology (Rockford, IL, USA). Standard Western blots were prepared and incubated overnight with specific primary antibodies against p-ATM (Ser1981), p53, p21, p16INK4A, phosphor-gamma-H2AX (Ser139) and β-actin. The immune complexes were detected by Immobilon Western Chemiluminescent HRP Substrate (Merck, DA, Germany) and exposed by an Azure c300 imaging system (Azure Biosystems, CA, USA).

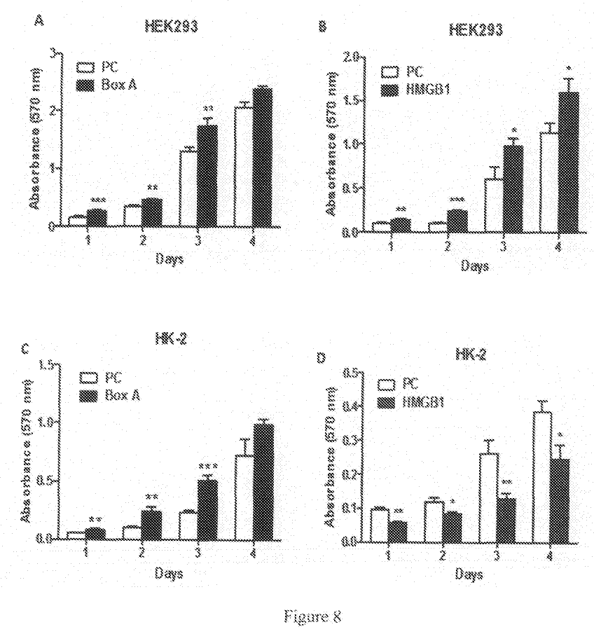

The cellular response to DNA damage is regulated by the DDR signaling pathway, which consists of protein kinase cascades that facilitate phosphorylation within the DDR network (10). To determine the effect of HMGB1 and Box A plasmids on DDR, the present disclosure evaluated the protein expression levels of γH2AX, p-ATM (ser1981), p53, p21, and p16INK4A (11,12). The present disclosure found that the expression levels of the DDR signaling pathway proteins were decreased in HMGB1- and Box A-transfected cells (FIG. 6).

### Example 7

The present disclosure revealed that Box A of HMGB1 facilitates cell proliferation. To investigate cell proliferation after transfection with HMGB1 and Box A plasmids, transfected cell lines were assessed after seeding every day for 4 days using MTT reagent (5 mg/ml) (Sigma-Aldrich®, Missouri, USA) and measured at 570 nm using a microplate reader (Bio-Rad, Hercules, CA, USA). A significantly higher rate of cell proliferation in HMGB1- and Box A-overexpressing HEK293 and HK-2 cells than in the control cells were observed (FIG. 7).

### Example 8

The present disclosure also hypothesized that overexpression of Box A and HMGB1 genes would increase cellular resistance to DNA damage. To prove this possibility, cells transfected with Box A of HMGB1, HMGB1 and scramble plasmids were treated with DNA-damaging agents, including H₂O₂ and methyl methane sulfonate (MMS). Particularly, the MTT assay was used to evaluate the cell survival rate under DNA-damaging agent treatment. Cells were seeded at 4,000 cells in 100 μl (40,000 cells/ml) in a 96-well plate. After plasmid transfection twenty-four hours later, cells were treated with medium containing an increased concentration of MMS (Sigma-Aldrich®, Missouri, USA) (0-2 mM) for one hour, and hydrogen peroxide (H₂O₂) (Sigma-Aldrich®, Missouri, USA) (0-250 μM) for 24 hours in a CO₂ incubator. Then, the media containing MMS or H₂O₂ was replaced with normal working media. Cell growth was measured with an MTT assay 48 hours after treatment. Data are presented as the percentage of cell survival, where the survival rates for the control (medium without DNA-damaging agents) were arbitrarily set to 100%. Treatment of cells with the DNA-damaging agents showed that the percentage of cell survival of overexpressed Box A and HMGB1 cells was significantly higher than that of scramble cells (FIG. 7 A-B). Interestingly, Box A overexpression protected cells better than HMGB1 (FIG. 7 A-B). Moreover, MTT and cell counting assays suggest that increased expression of Box A of HMGB1 in cells is an effective and safe method of DNA damage prevention.

### Example 9

The present disclosure hypothesized that overexpression of Box A and HMGB1 genes would improve healing process of individuals suffering from accelerated biological aging condition, such as patients of DM. The animal use protocol was approved by the Institutional Animal Care and Use Committee (IACUC) at the Faculty of Medicine, Chulalongkorn University (approval number: 006/2561 in September 2018). Male Wistar rats (6 weeks old, 150-180 g) were randomly divided into two groups and intraperitoneally injected with a single dose of 65 mg/kg body weight of STZ (Sigma-Aldrich®, Missouri, USA-Aldrich, USA), dissolved in 50 mM sodium citrate buffer (Alfa Aesar, USA), and 50 mM sodium citrate buffer (2 mL/kg body weight) (13). Seven days after STZ induction, the STZ-induced rats with a FBS greater than 250 mg/dL were designated as the diabetic group, whereas the rats with FBS lower than 150 mg/dL were considered as the nondiabetic group.

Two paired full-thickness excisional wounds were created at the dorsa of rats using an 8-millimeter biopsy punch and splinted with silicone rings (14). Diabetic and nondiabetic rats were further subdivided into three groups and treated with nanocoated Box A of HMGB1 plasmid, nanocoated PC, and NSS. The nanocoated pcDNA 3.1 (+) plasmid control was used as a nontreated control, and NSS was represented as a standard wound dressing in this study. The nondiabetic and diabetic wounds were dressed daily and treated with each type of intervention for 14 days. The wound area was measured at days 0, 3, 5, 7, and 14 after the treatment and reported as the % wound closure rate using the formula: the percentage of wound closure rate=[(wound area day 0−wound area day n)/wound area day 0]×100 (day n represented day 3, 5, 7, or 14). After 14 days of the complete healing process, all rats were sacrificed, and the areas of wounds were excised and immediately collected in 10% formalin buffer for histological and immunohistochemical for 8-OHdG determination.

After wound collection, the wounds were fixed in 10% neutral buffered formalin for at least 48 hours. Then, the tissues were dehydrated and paraffin-embedded before 3-μm thick tissue sectioning by a microtome. Subsequently, the tissue sections were stained with H&E and Giemsa for tissue histopathology and immune cell infiltration, respectively. The histopathological evaluation was blindly performed and interpreted by two pathologists. The tissue granulation and re-epithelization were investigated in the observed areas of healing wounds and reported as the overall histological score, including 1=normal tissue, 2=mature fibroblasts, 3=immature fibroblasts, 4=mild inflammation, and 5=granulation tissue.

Three micrometer paraffin-embedded sections were deparaffinized and then subjected to antigen retrieval by proteinase K (DAKO, CA) incubation for 2 min. Tissue sections were treated with a 1:8,000 dilution of polyclonal goat anti-8-OHdG (Merck Millipore), followed by an HRP-conjugated anti-goat secondary antibody (DAKO, CA). The wound sections were also counterstained with hematoxylin. The present disclosure tested the efficiency of Box A of HMGB1 plasmid-encapsulated Ca—P nanoparticle in facilitating healing process of a murine DM wound model. The results acquired from the experimented murine DM was reported in Table 1 below.

To investigate the effect of Box A of HMGB1 plasmid/Ca—P treatment on diabetic wound closure, splinted 8-mm excisional wounds were topically treated with either Box A of the HMGB1 plasmid, the PC or NSS once per day for 14 days. To deliver the plasmids into the target cells, each type of plasmid was coated with nanoparticle solution as done in previous study of Zhao et al. (2014) with some modifications before topical administration (15). The most effective ratio of the plasmid to nanoparticle solution for transfection was 5 μg plasmid in 100 μl nanoparticle solution. Briefly, the Ca—P nanoparticle solution was composed of 50 μl of a mixture of 0.5 M calcium chloride (CaCl2)) solution (Merck Millipore, USA) and 5 μg plasmid DNA, and 50 μl of a mixture of 0.01 M sodium carbonate (Na2CO3) solution (Merck Millipore, USA) and 0.01 M sodium dihydrogen phosphate monohydrate (NaH₂PO4·H2O) solution (Merck Millipore, USA) was prepared. The 3-molar ratio of CO32-/PO4 (31:1) was used. First, the plasmid DNA-calcium complex was prepared by mixing 16 μl of CaCl2) solution and plasmid DNA with the final volume adjusted to 50 μl using sterile dH2O. Then, the plasmid DNA-calcium complex was added to 50 μl of the mixture of Na2CO3 and NaH2PO4·H2O solution (16 μl) and sterile dH₂O (34 μl). The nanoparticle-coated plasmid solution was prepared prior to use.

TABLE 1
The body weight and fasting blood glucose level of nondiabetic
and diabetic rats at the end of the study. Seven days after
STZ induction, pretreatment fasting blood glucose levels were
measured; levels >250 mg/dL were defined as diabetic, and
rats meeting the criterion were included in the diabetic group.
Citrate buffer was injected into rats in the nondiabetic group
(FBS <150 mg/dL), which served as a control group. After
wounding, the diabetic wounds were treated daily with NSS, Nano-
PC, or Nano-Box A and compared to the untreated nondiabetic wound,
and post-treatment FBS was confirmed at the end of the study.

| | | | Post-treatment | | | |
|---|---|---|---|---|---|---|
| | Pre-treatment | | | | DM + | DM + |
| | Non- | | Non- | DM + | Nano- | Nano- |
| Group | DM | DM | DM | NSS | PC | Box A |
| Body | 251.7 ± | 186.0 ± | 277.5 ± | 192.8 ± | 190.2 ± | 195.2 ± |
| weight (g) | 18.6 | 17.0*** | 3.4 | 5.7*** | 7.3*** | 6.8*** |
| Fasting | 133.5 ± | 440.8 ± | 127.0 ± | 432.5 ± | 407.6 ± | 417.8 ± |
| blood | 14.2 | 21.9*** | 8.6 | 17.7*** | 25.1*** | 11.2*** |
| glucose | | | | | | |
| (mg/dL) | | | | | | |

Abbreviation: Non-DM; Non-diabetic group, DM; diabetic group, DM+NSS; diabetic wound treated with normal saline, DM+Nano-PC; diabetic wound treated with nanoparticle with plasmid control, DM+Nano-Box A; diabetic wound treated with nanoparticle with Box A plasmid. Data were represented as the means±SEM. *** P<0.001 significant difference, in comparison with the non-DM group.

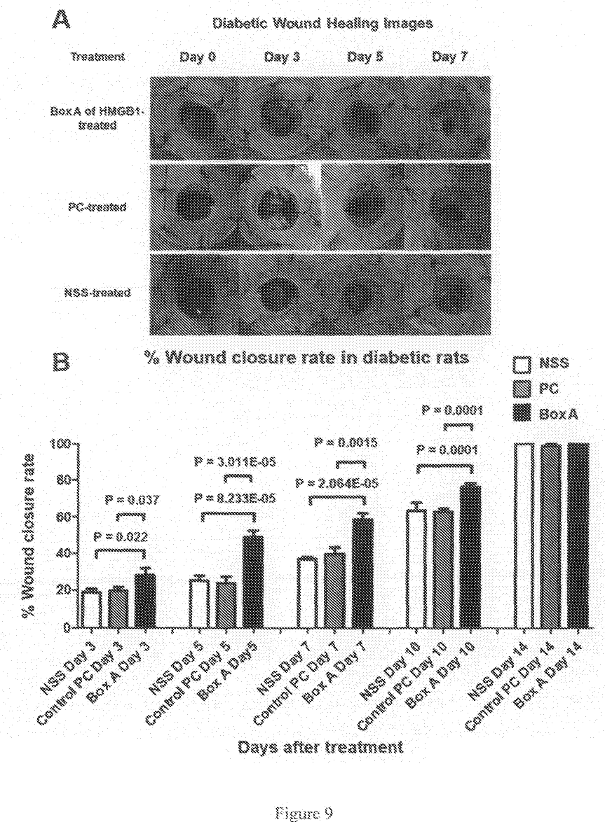

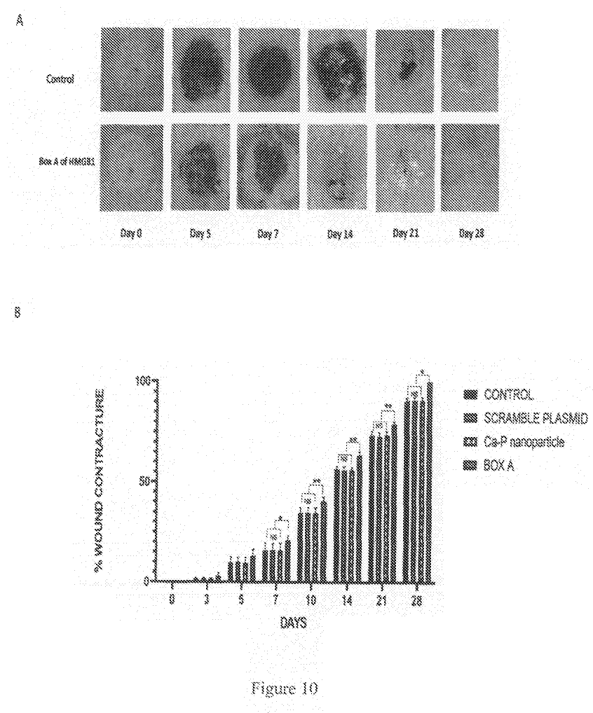

A representative image of the diabetic wounds at days 0, 3, 5, 7, 10, and 14 after wounding exhibited a smaller area of the diabetic wounds after treatment with Box A of the HMGB1 plasmid compared to either the PC or NSS treatment (FIG. 9A). Compared with the control-treated group, the treatment of Box A of HMGB1 plasmid revealed increased wound closure, especially during days 5 to 7 (P<0.0001) (FIG. 9B). Box A of HMGB1 treatment improved average histological score with high numbers of mature fibroblasts and less inflammation (overall grading) and significantly lower levels of 8-OHdG by immunohistochemical staining in diabetic wound sections than the levels in either the PC-treated or NSS-treated sections (P<0.001) (Table 2). These experiments supported that Box A of HMGB1 facilitated wound healing specifically to DM patients having a low level of Youth-DNA-GAPs.

TABLE 2
Histological parameters of diabetic wounds at day 14 after daily treatment
with NSS (N = 8), PC (N = 8), or Box A (N = 8). Histological
scores were graded and included overall grading (1 = normal
tissue, 2 = plenty of mature fibroblasts, 3 = plenty of
immature fibroblasts, 4 = mild inflammation, and 5 = granulation
tissue), fibroblasts (0 = absent, 1 = immature, and 2 =
mature), fibrosis (0 = absent, and 1 = present), and new
vessel formation and inflammatory infiltration (0 = absent,
1 = mild, 2 = moderate, and 3 = abundant). Anti-8-
OHdG staining was also evaluated (0 = absent, 1 = mild,
2 = moderate, and 3 = abundant).

| | NSS-treated | PC-treated | Box A-treated |
|---|---|---|---|
| Histological parameter | | | |
| Overall grading | 2.00 ± 0.00 | 2.80 ± 0.31 | 1.54 ± 0.14*†† |
| Fibroblasts | 1.00 ± 0.00 | 1.00 ± 0.00 | 1.53 ± 0.14*† |
| Fibrosis | 0.20 ± 0.20 | 0.00 ± 0.00 | 0.46 ± 0.14† |
| New vessels | 0.40 ± 0.24 | 1.00 ± 0.00 | 0.69 ± 0.13 |
| Immune cell infiltration | | | |
| Neutrophils | 0.00 ± 0.00 | 0.20 ± 0.16 | 0.07 ± 0.07 |
| Lymphocytes | 1.00 ± 0.00 | 1.00 ± 0.00 | 0.77 ± 0.12 |
| Macrophages | 0.80 ± 0.58 | 0.80 ± 0.40 | 0.54 ± 0.18 |
| Plasma cells | 1.00 ± 0.00 | 1.00 ± 0.00 | 0.76 ± 0.12 |
| DNA damage marker | | | |
| 8-OHdG | 2.43 ± 0.30 | 2.44 ± 0.20 | 1.44 ± 0.20**††† |

Abbreviation: NSS-treated; normal saline solution-treated group, PC-treated; plasmid control-treated group; Box A-treated; Box A of HMGB1 plasmid-treated group.
Data are presented as the means ± SEM.
*P < 0.05 and
**P < 0.01 significant difference, in comparison with NSS-treated group,
†P < 0.05 significant difference, in comparison with PC-treated group, and
††P < 0.01 and
†††P < 0.001 significant difference, in comparison with the PC-treated group.

### Example 10

The present disclosure hypothesized that overexpression of Box A and HMGB1 genes would improve healing process of individuals exposed to DNA damaging agents, such as excessive heat. The animal experimentation protocol followed the Guide for the Care and Use of Laboratory Animals published by the National Institutes of Health Guide for the Care and Use of Laboratory animals (NIH Publications No. 8023, revised 1978). All animal experiments were performed in accordance with the Animal Care and Use Committee of Chulalongkorn University (approval number: 003/2562 on March 2019). This study used One-way analysis of variance (One-way ANOVA) by program G*Power 3.1 for calculate sample size with alpha error=0.05, power=0.95, effect size f=1, number of groups=4, the result was about 32 wounds from the 16 rats used for the experiment (16). Sixteen male 8-week-old Wistar rats (150-180 g) were obtained from the Namura Laboratory Animal Center (Bangkok, Thailand). The rats were acclimated for seven days under a controlled 12-h light/dark cycle and fed standard chow and water ad libitum. To create second-degree burns, rats were anaesthetised with isoflurane and the dorsal skin was shaved. Two second-degree burn wounds were produced on the back of each rat using a 10 mm wide aluminum rod heated to 100° C. The rats were further divided into four groups and the wounds were treated daily with normal saline solution (NSS), scramble-flag plasmid-treated group (plasmid control group), calcium phosphate nanoparticle-treated group (control group) and Box A plasmid-treated group. Nano-coated pcDNA 3.1 (+) (scramble) plasmid control and Calcium phosphate Nanoparticle group were used as a non-treated control and NSS was represented as a standard wound dressing in this study. Wound areas were measured 0, 7, 14, 21 and 28 days after wounding using the NIH ImageJ analyzing tool and reported as the percentage wound contracture relative to day 0. All rats were euthanized after 28 days and the wound tissues were excised and immediately collected in 10% formalin buffer for further assessment. In the group of Box A of HMGB1 protein demonstrated a significant burn wound closure rate, starting from day 7th until day 28th after wounding, compared to the wound closure rate of the normal saline, scramble plasmid and calcium-phosphate nanoparticle treated group (FIG. 10) especially during days 10 to 21 (P<0.001). In contrast, no significant difference between the plasmid control-treated wounds, calcium-phosphate nanoparticle and normal saline treated wounds was observed at each time point.

### Example 11

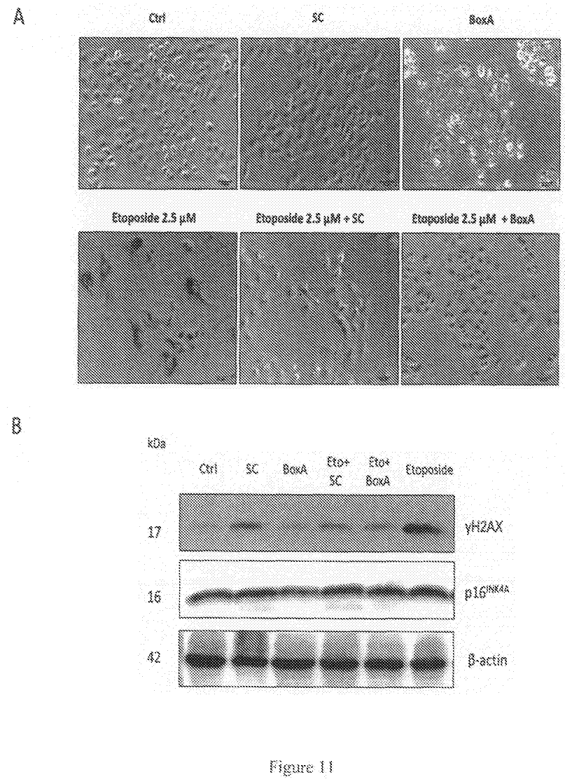

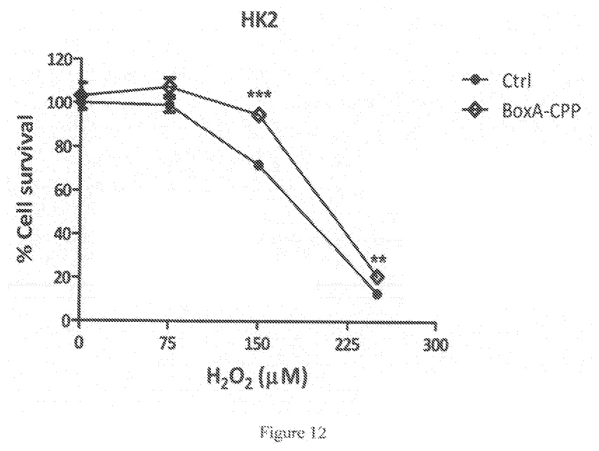

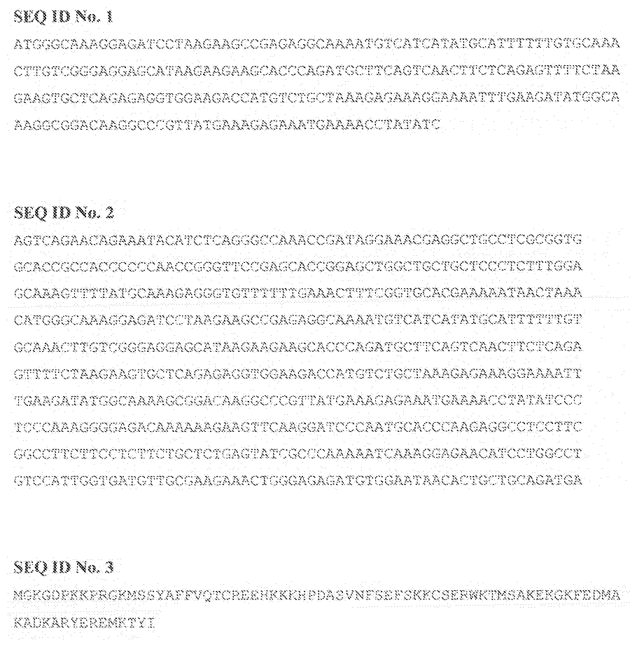

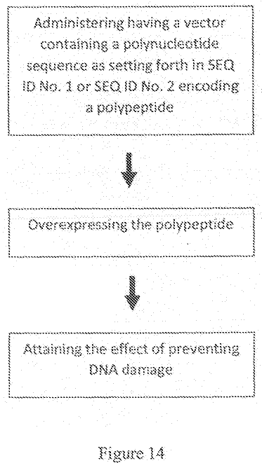

The present disclosure revealed the efficiency of Box A of HMGB1 in rejuvenating biological aging cells by showing the reverse aging effect of BoxA on cellular senescence of cells induced by 2.5 μM of etoposide. HK2 cells were pretreated with 2.5 μM of etoposide for 72 h. Etoposide was used to induced cellular senescence as previously described (17). After 72 h, cells were transfected with BoxA of HMGB1 or PC plasmids (final concentration of plasmids, 2,500 ng/ml) using Lipofectamine 3000 and incubated for 48 h. Representative cell images showed that etoposide pretreatment affected to cell density with characteristics of senescent cells, such as an enlarged and flattened cell shape, loss of proliferative potential compared to control, scramble and BoxA transfected group. β-galactosidase (SA-β-gal) was evaluated as previously described using SA-β-gal staining kit (Cell Signaling Technology, Beverly, MA, USA) according to the manufacturer's instructions (18). The SA-β-gal-positive staining cells have been significantly reduced after transfection with BoxA plasmid whereas β-gal-positive cells remained at a high level after reversed with scramble plasmid (FIG. 11 A).

Furthermore, the present disclosure examined the protein expression of p16^{INK4A} which is a senescence-associated cell cycle inhibitor and 7H2AX in HK2 cells (19). The results showed that 2.5 μM etoposide increased the expression of p16^{INK4A} and γH2AX compared with PC, BoxA of HMGB1 transfected cells, and control. The level of p16^{INK4A} and γH2AX of cells received BoxA HMGB1 after etoposide treatment was significantly lower than the etoposide treated group (FIG. 11B).

### Example 12

Genomic stabilization role of Box A of HMBG1 peptide with peptide transfection system such as cell-penetrating peptide (20). Here, IMT-P8 was cell-penetrating peptide (21). To evaluate the effect of IMT-P8-BoxA peptide (Genscript, Piscataway, NJ, USA), cells were treated with medium containing IMT-P8-BoxA peptide at concentration 0.25 μM for 2 h. After that, cells were washed with 1×PBS and retreated with hydrogen peroxide (H₂O₂) (Sigma Chemical, St. Louis, MO, USA) for 24 h. Then, cells were replaced with normal medium and incubated for 48 h at 37° C. Finally, cells viability was measured with microplate reader (Thermo Fisher Scientific, Waltham, MA, USA). FIG. 19 shows that treated cells with IMT-P8-BoxA peptide increasing the percentage of cell survival after incubating with H₂O₂ than control cells.

The present disclosure reported the first genomic stabilizing biomarker, Youth-DNA-GAPs, and medicine, Box A of HMGB1, that is promising for monitoring and preventing genomic instability, which causes health deterioration in a number of disease conditions. First, we reported that Phy-RIND-EDSB or Youth-DNA-GAP is a unique type of epigenomic marker in humans that decreases in elderly and DM patients. Second, we proved that HMGB1 produced Youth-DNA-GAPs. Third, the Youth-DNA-GAPs increase the stability of the DNA strand in long-distance cis so Box A of HMGB1 can reduce endogenous DNA damage as well as the DDR, and increase cellular resistance to DNA-damaging agents. Box A of HMGB1 was used to fix wound healing delays in DM rats and burn. Finally, Box A of HMGB1 was used to rejuvenate senescence cells.

A previous study showed that the inverse association between Youth-DNA-GAPs and biological aging was very strong in yeast (4). Here, in humans, a strong association was also demonstrated in elderly individuals and DM patients, both of whom are known to have biologically aging DNA. DNA damage is commonly found in the elderly and age-associated NCDs (22,23). Therefore, Youth-DNA-GAPs may be an effective biomarker for determining the biological age of NCD patients. Here the present disclosure showed that the genome stabilization function of HMGB1 is mediated by Youth-DNA-GAPs. HMGB1 possesses the ability to bend DNA and stabilize double-stranded DNA against denaturation (24). These two properties supported findings of the present disclosure that Youth-DNA-GAPs are produced by HMGB1. Moreover, similar to EDSBs generated by topoisomerase (25), Youth-DNA-GAPs may stabilize the eukaryotic genome by relieving torsion force and consequently stabilizing double-stranded DNA against denaturation. Box A of HMGB1 not only reduces endogenous DNA damage but also increases cellular resistance to DNA-damaging agents. Relieving the torsion force by the gaps structure of Youth-DNA-GAPs should increase stability of DNA. It is interesting that Box A stabilized the genome more efficiently than the whole HMGB1 protein. One possible explanation is that HMGB1 is a multifunctional protein in both the nucleus and extracellularly (26). The presence of Box B and C terminals should divide the molecule to play other roles, such as inflammation. Nevertheless, excluding the Box B and C terminals helped specify the Box A of HMGB1 role to genomic stabilization.

Youth-DNA-GAPs were known to be reduced by global DSB repair after a DSB induction event (4). Therefore, delay genomic instability mechanism of cell exposed to DNA damaging agents may involve Youth-DNA-GAP reduction and this may explain why Box A of HMGB1 facilitated burn wound healing. The present disclosure showed that molecularly engineered Box A of HMGB1 can stabilize eukaryotic genome through production of Youth-DNA-GAPs. The present disclosure demonstrated that the genome stabilizing property of Box A of HMGB1 leads to numerous applications including reduction of endogenous DNA damage, reduction of DDR, increase cellular resistance to DNA damaging agent, promotion of cell growth, promotion of tissue healing process of individuals with DM, promotion of tissue healing process of burn wound injury and rejuvenation of aging cells. If any other NCDs caused by low levels of Youth-DNA-GAPs, the present disclosure may potentially use Box A of HMGB1 as a genomic stabilizing molecule for treating such NCDs.

It is to be understood that the present disclosure may be embodied in other specific forms and is not limited to the sole embodiment described above. However, modification and equivalents of the disclosed concepts such as those which readily occur to one skilled in the art are intended to be included within the scope of the claims which are appended thereto.

1-9. (canceled)

10. A vector capable of expressing a peptide in cells for preventing damage of DNA in a nucleus of the cells, the vector comprising a polynucleotide sequence as setting forth in SEQ ID No. 1 encoding for the peptide, wherein the peptide is configured to increase production of Phy-RIND-EDSBs in the nucleus.

11. The vector of claim 10, wherein the cells are aging cells with Youth-DNA-GAPs lower than 0.3% EDSB PCR of a control and/or an accumulation of senescence-associated β-galactosidase cells more than 50%.

12. A method for rejuvenating DNA and/or preventing DNA damage within a nucleus of cells, the method comprising:
transfecting the cells with a vector containing a polynucleotide sequence as setting forth in SEQ ID No. 1 encoding for the peptide into the cells, the vector being configured to express the encoded peptide within the cells subsequent to the transfecting step; and
producing Phy-RIND-EDSBs in the nucleus of the cells for prevention of DNA damage, wherein the cells are aging cells with Youth-DNA-GAPs lower than 0.3% EDSB PCR of a control and/or an accumulation of senescence-associated β-galactosidase cells more than 50%.

13. The method of claim 12 further comprising overexpressing the peptide encoded by the polynucleotide sequence resided in the transfected vector.

14. The method of claim 12, wherein the prevention of DNA damage is obtained by way of increasing cellular resistance against DNA damaging agent.


SEQUENCE LISTING (3 sequences)

SEQ ID NO: 1 - DNA, 237 bp, Homo sapiens
  Box A of high mobility group box 1 (HMGB1) (1)..(237)
atgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatc

SEQ ID NO: 2 - DNA, 600 bp, Homo sapiens
  high mobility group box 1 (HMGB1) mRNA (1)..(600)
agtcagaacagaaatacatctcagggccaaaccgataggaaacgaggctgcctcgcggtggcaccgccaccccccaaccgggttccgagcaccggagctggctgctgctccctctttggagcaaagttttatgcaaagagggtgttttttgaaactttcggtgcacgaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatga

SEQ ID NO: 3 - protein, 79 aa, Homo sapiens
  Box A of high mobility group box 1 (HMGB1) (1)..(79)
MGKGDPKKPRGKMSSYAFFVQTCREEHKKKHPDASVNFSEFSKKCSERWKTMSAKEKGKFEDMAKADKARYEREMKTYI